(19) United States
(12) Patent Application Publication
(10) Pub. No.: US 2002/0136722 A1
(43) Pub. Date: Sep. 26, 2002

(54) Vaccination method

(76) Inventor: Andrew William Heath, Sheffield (GB)

Correspondence Address: CROWELL & MORING LLP, INTELLECTUAL PROPERTY GROUP, P.O. BOX 14300, WASHINGTON, DC, 20044-4300, US

(21) Appl. No.: 10/056,058

(22) Filed: Jan. 28, 2002

Related U.S. Application Data
(63) Continuation-in-part of application No. 09/938,477, filed on Aug. 24, 2001.
Application No. 09/938,477 is a continuation of application No. 08/878,348, filed on Jun. 18, 1997, now abandoned.

(51) Int. Cl. A61K 39/395

(52) U.S. Cl. 424/144.1

(57) ABSTRACT

The invention relates to adjuvants comprising CD40 ligands crosslinked to antigens, wherein the adjuvants have reduced toxicity and adjuvants comprising CD40 ligands crosslinked to viral antigens.

### CROSS REFERENCE TO RELATED APPLICATIONS

This application is a continuation-in-part of co-pending U.S. patent application Ser. No. 09/938,477, filed Aug. 24, 2001, which in turn in a continuation of U.S. patent application Ser. No. 08/878,348, filed Jun. 18, 1997.

### BACKGROUND OF THE INVENTION

The invention relates to a method of manufacture and a system for the production of novel human or animal vaccine; and also a novel human or animal vaccine.

It is known that the immune system works on the basis of recognition and thus the ability to distinguish between self and non-self. Recognition of non-self, or invading material, is followed by a sequence of steps that are designed to kill or eliminate the non-self material. As knowledge of the immune system grows and molecular biological techniques advance it has become possible to advantageously manipulate the various steps in an immune response in order to enhance the nature of that response. Thus, for example, it has become possible to manufacture a wide range of vaccines using recombinant material and thus manufacture a range of vaccines which were not previously available either because the relevant material was not obtainable or had not before been produced.

The immune system is made up of lymphocytes which are able to recognise specific antigens. B lymphocytes recognise antigens in their native conformation through surface immunoglobulin receptors, and T lymphocytes recognise protein antigens that are presented as peptides along with self molecules known as MHC, on the surface of antigen presenting cells. There are a variety of antigen presenting cells including B lymphocytes. T lymphocytes may be further subdivided into cytotoxic T lymphocytes, which are able to kill virally infected “target” cells, and T helper lymphocytes. T “helper” lymphocytes are able to help B lymphocytes to produce specific antibody, or to help macrophages to kill intracellular pathogens.

Bacterial infections caused by encapsulated bacteria are a major world health problem. The species Streptoccocus pneumoniae, Haemophilus influenzae and Neisseria meningitidis are difficult to vaccinate against due to the thymus independent nature of the major surface antigens, the capsular polysaccharides.

T-cell independent antigens present particular problems regarding the development of effective vaccines. Antibody production is low and is not normally boosted by re-immunization. The antibody isotypes are restricted to the IgM and other isotypes are generally of a low affinity for a specific antigen.

A major problem lies in the response of young children to T-cell independent vaccines. These individuals are amongst the most vulnerable to the aforementioned bacterial infections. Over 80% of childhood pneumococcal infections occur in infants under the age of two. Coincidentally this age group responds most poorly to T-cell independent antigens.

### SUMMARY OF THE INVENTION

T-cell independent antigens are much more effective at eliciting high titre, high affinity antibody responses. This comes about because T-lymphocyte help to B-lymphocytes is elicited during the immune response to these antigens. B-lymphocytes bind to antigen through their specific antigen receptors which leads to partial activation. If the antigen is a protein the B-lymphocytes take up and process the antigen to peptides which are expressed on the cell surface along with MHC class II molecules. The MHC class II/peptide complex is then recognised by specific T-lymphocytes. Upon this recognition the T-lymphocytes give “help” to the B-lymphocytes, and this “help” along with the initial signal through the antigen receptor results in increased B-lymphocyte proliferation, isotype switching and possibly also to increased affinity antibody being eventually produced through somatic hypermutation in the antigen receptor genes. T-cell independent antigens are invariably not protein in composition and cannot therefore be processed and presented by B-lymphocytes via MHC molecules. This failure in antigen presentation results in low T-cell recognition of the antigen thereby resulting in no T-cell help.

T-cell help to B-cells has two components which together with signals through the antigen receptor lead to B-lymphoctye proliferation and antibody production.

1. Cell-cell mediated activation.

2. Cytokine activation.

In vitro experiments have shown that resting B-cells can be stimulated to proliferate after exposure to isolated membranes from activated T-cells. The basis for this phenomenon has been determined. Following T-cell activation a 39 kDa (CD154) T-cell specific cell surface protein is induced. This ligand has been identified as the target of the B-cell cell surface receptor CD40 and binding of CD154 to CD40 is the major component of T-lymphoctye help to B-lymphocytes.

Further evidence for the involvement of CD40 and CD154 comes from experiments in which host cells transfected with the cDNA encoding the CD154 protein can induce proliferation of B-cells in the presence of added cytokines. In addition, patients with the congenital disease X-linked hyper IgM syndrome, who fail to switch antibody isotypes have been shown to have various mutations in the gene encoding the CD154 protein resulting in failure to activate the B-cells via CD40. The CD40-CD154 interaction has also been shown to be an important element in immune responses to T-cell dependent antigens in ‘knock-out’ mice.

The other important element in B-cell activation via T-cell help involves cytokine function. Although isolated membranes from activated T-cells can induce B-cell proliferation this effect can be enhanced by the presence of cytokines. Furthermore cytokines have a major role in switching of antibody isotypes. In particular IL4, interferon γ and transforming growth factor beta (TGF β) are of importance. IL4 induces IgG1 and IgE, IFNγ induces IgG2a and TGFβ induces IgA and IgG2b. In addition IFNγ is probably responsible for the switching to IgG3 which is seen naturally in responses to T-cell independent antigens. However ligation of CD40 does not induce appreciable Ig secretion on its own, but CD40 ligation (including via T-cell membranes) seems to prepare cells for differentiation which can be induced efficiently by IL4 and IL5.

Finally T-cell help has a major influence on somatic hypermutation which results in the selection of B-cell clones that produce high affinity antibodies.

From this description it may be surmised that T-cell independent production of antibodies by B-cells is compromised due to the lack of help offered by T-helper lymphocytes through activation via CD40 and through the influence of cytokines produced by the T-helper cell.

It is therefore an object of this invention to provide a means of activating B-cells to proliferate and produce the full range of antibody isotypes of high titre in response to T-cell independent, as well as T-cell dependent antigens.

It is a further object of this invention to use T-cell independent and/or dependent antigens to produce effective vaccines that offer high titre, high affinity antibodies to protect individuals from infection.

It is yet a further object of the invention to provide a safe immunological adjuvant of low toxicity for use in a vaccine and also for use in enhancing the immune response to T-cell independent and/or dependent antigens.

It is yet a further object of the invention to provide a system for the production of the vaccine of the invention.

In its broadest aspect the invention concerns the provision of a means for activating the CD40 receptor on a B-lymphoctye, ideally the means comprising an adjuvant which is adapted to activate said receptor, either directly or indirectly. More preferably the invention concerns a ligand which binds to the CD 40 receptor on a B-lymphocyte and brings about the activation of same.

According to a first aspect of the invention there is provided an adjuvant which is adapted to stimulate a B-lymphocyte cell surface receptor, CD40.

According to a second aspect of the invention there is provided a vaccine suitable for enhancing T-cell independent and T-cell dependent immunity comprising a T-cell dependent and/or independent antigen, or part(s) thereof, and an associated adjuvant which is adapted to stimulate a B-lymphocyte cell surface receptor, CD40.

Reference herein to the term vaccine is intended to include a wide variety of vaccines including, but not limited to, contraceptive vaccines, immunotherapy vaccines and prophylactic or therapeutic vaccines.

Reference herein to T-cell independent immunity includes reference to an immune response which operates wholly or largely independently of T-cells, for example, because existing T-cells are not activated; or because existing T-cells are not functional or immune suppressed through disease or exposure to chemicals, radiation or any other means.

To by-pass or mimic the effects of T-cells help we propose a vaccine which ensures that all B-cells receiving a signal through their specific antigen receptors also receive a signal through CD40, mimicking or improving upon that which would be received during natural T-cell help. This would be achieved, ideally, by ensuring that a CD40 binding moiety were closely associated with the vaccine antigen. This could be through co-administration of the CD40 stimulating moiety with the appropriate T-cell independent and/or dependent antigen, or preferably through covalent linkage, or co-entrapment on/in a carrier system.

The vaccine involves ideally the conjugation of the antigen to a CD40 ligand such as an anti CD40 antibody, or part thereof, followed by immunisation of a human or animal. It should be apparent to those skilled in the art that this methodology may also be applied to any antigens, but in the instance of T-cell dependent antigens could be of particular relevance to those individuals that are immune suppressed and therefore lack T-helper lymphocytes (e.g. AIDS patients).

In a preferred embodiment of the said invention said antigen is soluble and ideally a protein or a polysaccharide.

Ideally stimulation of CD40 is via binding of said adjuvant, or part thereof, to at least a part of CD40. In a preferred embodiment of the invention said antigen and adjuvant are bound or cross-linked together.

More preferably said adjuvant is an antibody, either polyclonal or monoclonal, but ideally monoclonal, which is adapted to bind to said CD40. More ideally still said antibody is humanised.

In a preferred aspect of the invention said antibody may be whole or, alternatively, comprise only those domains which are effective at binding CD40 and in particular selected parts of CD40.

In another embodiment of the invention, said adjuvant is a natural ligand of CD40, the T-cell specific CD154 cell surface antigen, ideally produced as a recombinant protein, or a CD40 binding portion of the CD154 protein, or indeed any other ligand, or part thereof, that binds CD40 or part thereof.

In a further embodiment, the CD40 ligand may not be a naturally occurring CD40 ligand but represent an agent that due to its biochemical characteristics has an affinity for CD40.

In its broadest context, reference herein to the term adjuvant includes reference to any string of amino acids or ligand which is selected so as to bind to at least a part of CD40.

In a preferred aspect the recombinant vaccine antigen (when a polypeptide) and the adjuvant will be produced as a chimeric fusion protein.

It will be apparent to those skilled in the art that the said antigen may be a T-cell independent antigen and thus any antigen which is capable of eliciting a T-cell independent response.

Alternatively, the antigen may be a T-cell dependent antigen and thus any antigen that is capable of eliciting a T-cell response.

It is apparent from the above that any antigen may be selected for use in the vaccine of the invention—the precise nature of which will depend on the “disease” that the individual is to be immunised against and/or in some circumstances, the immune status of an individual to be vaccinated.

Ideally said antigen and/or adjuvant is in the form of an immunostimulating complex, or liposomes or biodegradable microspheres, so increasing the association between antigen and CD40 binding moiety.

Alternatively said vaccine comprises an emulsion of the antigen and adjuvant ideally in oil.

In a preferred embodiment of the invention at least one selected cytokine may be included in and/or coadministered in/with said vaccine.

According to a third aspect of the invention there is provided an adjuvant for enhancing T-cell independent immunity wherein said adjuvant comprises an agent adapted to stimulate a B-lymphocyte surface receptor, CD40.

Preferably said stimulation of said CD40 is via binding of said adjuvant, or part thereof, thereto.

Ideally, said adjuvant is an antibody, either polyclonal or monoclonal, but ideally monoclonal, which is adapted to bind to said CD40. More ideally still said antibody is humanised.

In a preferred embodiment of the invention said antibody may be whole or, alternatively, comprise only those domains which are effective at binding CD40, and in particular selected parts of CD40.

In this aspect of the invention said adjuvant is co-administered with either said T-cell independent antigen that is effective at eliciting a T-cell independent immune response of a T-cell dependent antigen that is effective at eliciting a T-cell response. This will be dependent upon the nature of the “disease” against which the individual is to be immunised and/or the immune status of the individual.

More preferably further still said adjuvant is co-joined to said T-cell independent antigen or said T-cell dependent antigen.

In a yet further preferred embodiment said adjuvant is co-administered with at least one cytokine.

According to a fourth aspect of the invention there is provided a method for the manufacture of a novel vaccine capable of enhancing T-cell independent immunity or T-cell dependent immunity which method comprises the selection of a suitable T-cell dependent and/or independent antigen, or part(s) thereof, and association or combination of said antigen with an adjuvant wherein said adjuvant is adapted to stimulate a B-lymphocyte receptor, CD40.

According to a fifth aspect of the invention there is provided a method for the manufacture of a novel vaccine capable of enhancing T-cell independent immunity which method comprises the selection of a suitable T-cell dependent and/or independent antigen, or part(s) thereof, and association or combination of said antigen with an adjuvant wherein said adjuvant is adapted to stimulate a B-lymphocyte receptor, CD40.

In a yet further preferred method of the invention said adjuvant is recombinantly manufactured.

In a yet further preferred embodiment of the method of the invention said antigen and adjuvant are bound or cross-linked together.

The major T-independent antigens used in vaccines are bacterial capsular polysaccharides. In a preferred embodiment or method of the invention one will therefore purify polysaccharide antigens and crosslink them to a CD40 binding moiety. A commonly used technique for the crosslink of polysaccharide to protein is carbodiimide coupling. However a number of heterobifunctional cross-linking agents are commercially available for both protein-protein and protein-carbohydrate cross-linking. Heterobifunctional cross-linking agents have the advantage that they favour protein-carbohydrate cross-links thereby maximising the yield of adjuvant coupled to antigen.

Preferably said stimulation of said CD40 is via binding of said adjuvant, or part thereof, thereto.

Ideally, said adjuvant is an antibody, either polyclonal or monoclonal, but ideally monoclonal, which is adapted to bind to said CD40. More ideally said antibody is humanised.

In a preferred method of the invention one adds at least one cytokine to said vaccine.

According to a further aspect of the invention there is provided a system for the manufacture of a vaccine capable of enhancing T-cell independent or T-cell dependent immunity which system comprises a cell expressing a selected T-cell dependent and/or independent antigen, or part(s) thereof, and also an adjuvant capable of stimulating a B-lymphocyte receptor, CD40.

According to a yet further aspect of the invention there is provided a system for the manufacture of a vaccine capable of enhancing T-cell independent immunity which system comprises a cell expressing a selected T-cell dependent or independent antigen, or part(s) thereof, and also an adjuvant capable of stimulating a B-lymphocyte receptor, CD40.

More preferably still both said antigen (when a polypeptide) and said adjuvant are adapted so as to be secreted from said cell. This may be undertaken by providing both the antigen and adjuvant with secretion signals or providing for the production of a single piece of material comprising both the antigen and the adjuvant and having a single secretion signal associated therewith. It will be evident that in the former instance the said antigen and adjuvant will be found in associated or unbound or uncross-linked manner in the supernatant of the system and in the latter instance said antigen and adjuvant will be co-joined in the supernatant of the system.

Preferably said stimulation of said CD40 is via binding of said adjuvant, or part thereof, thereto.

Ideally, said adjuvant is an antibody, either polyclonal or monoclonal but ideally monoclonal, which is adapted to bind to said CD40. More ideally said antibody is humanised.

In a preferred aspect of the invention said antibody may be whole or, alternatively comprise only those domains which are effective at binding CD40, and in particular selected parts of CD40.

It will be apparent from the above that the invention is based upon the realisation that immune responses, whether to a T-cell independent or a T-cell dependent antigen, can be enhanced by stimulating the B-cell CD40 receptor using any suitable means.

According to a yet further aspect of the invention there is provided a nucleic acid molecule encoding any one or more of the aforementioned embodiments of the invention.

In this last aspect of the invention said nucleic acid molecule may be administered, conventionally, to an individual or animal to be treated so that the adjuvant and ideally also the antigen of the vaccine may be manufactured in vivo.

### BRIEF DESCRIPTION OF THE DRAWINGS

The invention will be described in further detail by way of illustrative experiments with reference to the following figures in which:

FIG. 1: Shows CD40 antibody induced enhanced, class switched antibody responses to PS3 (type 3 pneumococcal polysaccharide) (A) and increased total serum immunoglobulin (B). BLAB/c mice (6-10 weeks old) were injected i.p. with 20 ng of PS3 and 500 μg of 1C10, 4F11 (anti-mouse CD40) or isotype control antibody GL117. Sera were obtained days 7, 14 and week 14 after injection. The IgM and IgG isotype mean logarithmic titres are shown when they were maximal, respectively, day 7 and day 14 after injection. All negative results were given a logarithmic titre of 20, the lowest dilution used. * indicates statistical significance compared with the relevant GL117 control (Student's T test p<0.05);

FIG. 2: Shows antibody responses to other pneumococcal polysaccharides are also enhanced by CD40 antibody. IgM and IgG responses to types 8, 4, 12 and 19 W. pneumoniae capsular polysaccharides in mice immunised with the 23 capsular polysaccharides in Pneumovax II (Merck Sharp and Dohme, USA) and either the CD40 antibodies 4F11, 1C10 (anti-mouse CD40) or control antibody GL117. Groups of five BALB/c mice, were injected i.p. with either 500 μg of 1C10, 4F11 or control antibody GL117. These mice failed to respond to co-administered keyhole limpet haemocyanin nor were any CD4+ splenocytes discernable on FACS by FITC anti CD4 (data not shown). Sera were obtained on day 14 after injection. All negative results were given a logarithmic titre of 20, the lowest dilution used. All 1C10 responses were significantly different from the relevant GL117 control (Student's T test p<0.05);

FIG. 3 shows that the mechanism of 1C10 action is CD4+ cell independent. PS3 specific antibody logarithmic titres induced in CD4+ depleted BALB/c mice treated i.p. with 20 ng of PS3 and 500 μg of 1C10, 4F11, or control antibody GL117. These mice failed to respond to co-administered keyhole limpet haemocyanin nor were any CD4+ splenocytes discernable on FACS by FITC anti-CD4. Sera were obtained on day 14 after injection. All negative results were given a logarithmic titre of 20, the lowest dilution used. All 1C10 responses were significantly different from relevant GL177 control (students t test p<0.05);

FIG. 4: Shows CD40 antibodies induce responses to PS3 in normally unresponsive xid mice (A). Enhanced responses in BALB/c mice provide protection against S. pneumoniae challenged 9 months after treatment (B). (A) PS3 specific antibody responses in CBA/N (xid) mice injected with 20 ng of PS3 and 1C10, GL117 and/or control CBA/ca mice with 1C10 and GL117. The IgM and IgG isotype logarithmic titres shown are when they were maximal, respectively, day 7 and day 14 after injection. All negative results were given a logarithmic titre of 20, the lowest serum dilution uses. * indicates statistical significance compared with the relevant GL117 control (Student's T test p<0.05) (B). Percentage survival in BAB/c mice challenged with S. pneumoniae type 3, but administered 9 months previously with 20 ng PS3 and 500 μg of 1C10, GL117 or PBS. Survival in the 1C10 group was significantly enhanced compared to the control groups (p<0.05 χ² test);

FIG. 5: Shows the effect of a mixture of 10 μg biotin anti-CD40 or isotype control antibody with 10 μg avidin prior to i.p. immunisation of BALB/c mice. It can be seen that primary antibody responses to avidin conjugated to biotinylated CD40 antibodies are enhanced. BALB/c mice were immunized with either 10 μg of control IgG2a, 10 μg of avidin conjugated to anti CD40 monoclonal antibody 4F11, 10 μg of a combination of avidin conjugated to anti-CD40 antibodies 4F11 and 1C10 or 10 μg of non-conjugated avidin. Antibody responses against avidin were measured by ELISA at 10 days post-immunisation;

FIG. 6: Shows secondary antibody response to avidin alone following primary immunisation with avidin conjugated to anti CD40 antibodies 4F11 and 1C10. Experimental details are essentially as described in FIG. 5, except that mice received an immunisation with 10 μg avidin alone one month after primary immunisation as in FIG. 5, mice were bled 10 days after this second injection and antibody responses measured by ELISA;

FIG. 7 shows spleen weights of mice 5 days after injection with anti-CD40 or an isotype control antibody at various doses;

FIG. 8 shows total serum immunoglobulin levels 10 days after anti-CD40 administration;

FIG. 9 shows the antibody response to 10 μg ovalbumin in BALB/c mice induced by co-administration of ovalbumin with anti-CD40 or control antibody at doses from 500 μg to 0.1 μg;

FIG. 10 is a FACS of CD40 transfected fibroblast cells bound by influenza specific CD40 mAb which shows the binding of influenza-mAb to CD40 transfected L cells (anti-influenza detection). The grey, filled histogram represents background binding; the thick solid line represents binding of influenza IC-10, and the thin, broken line represents binding of influenza-GL117; and

FIG. 11 is a graph of the survival rate of mice immunised with HSV:CD40 conjugates after challenge with HSV which shows the enhanced protection against Herpes simplex virus infection induced by an anti-CD40/peptide conjugate. Groups of 5 C57/B16 were immunised with 10 μg of conjugated peptide (1C10 and control GL117), peptide alone, peptide mixed with 1C10 (at 100 μg mAb and 50 μg pHSV-CTL or equivalent amount to conjugates). Five days after immunisation, the animals were challenged with 1.9×10⁶ PFU of HSV (333 strain) via the i.p. route.

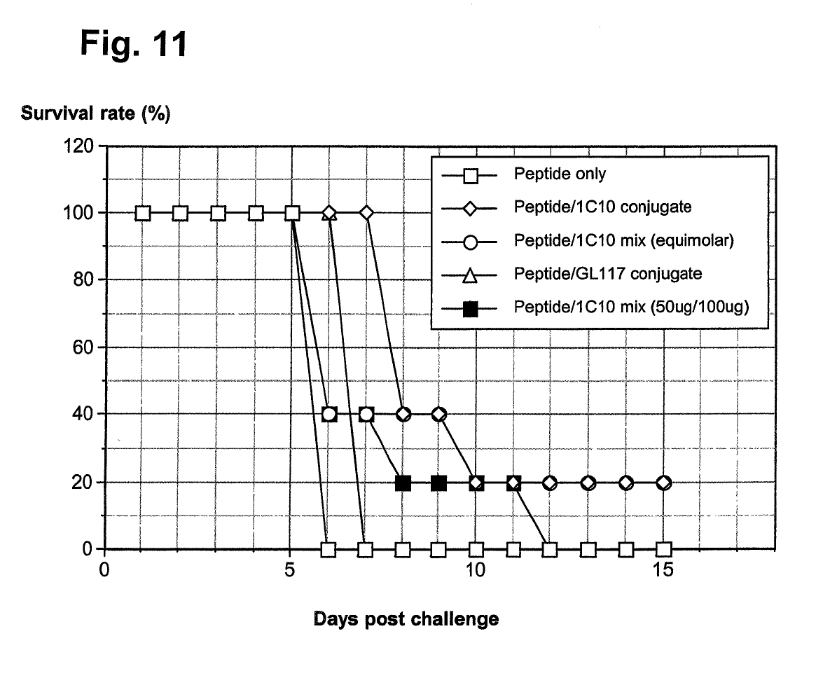

Materials and Methods

Mice

The mice used were BALB/c mice (in house), CBA/ca and CBA/N (xid) mice (Harlan-Olac). They were 6-12 weeks old at the start of the experiments. The pneumococcal capsular polysaccharides type 1, 3, 4, 8, 12, 13, 19 and 23 were obtained from ATCC, USA, pneumococcal cell wall polysaccharide from Statens Serum institute, Denmark and Pneumovax II vaccine from Merck Sharp and Dohme, USA. Avidin was purchased from Sigma (Poole, Dorset). Biotinylated and non-biotinylated anti-CD40 antibodies were purified from hybridoma supernatants in house and biotinylated in house were necessary using standard reagents (Pierce).

Conjugation of Anti-CD40 mAb to OVA

The anti-CD40 antibody, 1C10, along with its isotype matched control antibody (GL117) were conjugated to imject maleimide activated ovalbumin (Pierce, Rockford, Ill.) using N-succinimidyl S-Acetylthioacetate (SATA, also obtained from Pierce) as previously described by Baiu et al (1999). J. Immunol. 162: p. 3125-3130.

Briefly, antibody was dialysed against conjugation buffer (50 mM phosphate buffer containing 1 mM EDTA, pH 7.5) and concentrated by centrifuge filtration to 5 mg/ml. Immediately prior to use 6.5 mg of SATA was dissolved in 0.5 ml of DMSO. 1 ml of each of the antibody solutions were then incubated with 10 μl of SATA for 30 min at RT. Unbound SATA was removed from the solution by extensive washing through a 30 KDa cut-off centrifugal filter. Introduced sulfhydryl groups were deprotected by incubation of the reaction solution with 100 μl/ml of 0.5M hydroxylamine (in 50 mM phosphate, 25 mM EDTA, pH7.5) for 2 hr at RT. The solution was then diluted in 0.1M sodium phosphate, 0.15M NaCl, 0.1M EDTA containing the Imject maleimide activated OVA at a weight:weight ratio of antibody to OVA of 1:1.5. This reaction was allowed to proceed for 90 min at RT and was stopped by the addition of 2-ME to a final concentration of 10 mM. Conjugated OVA-mAb was separated from unconjugated reagents by extensive washing with PBS through a 300 KDa cut-off centrifuge filter. Concentration of conjugated mAb was determined by Bradford's reagent technique. The antibody-OVA product was filter sterilised and stored at 4° C. until required.

The size of mAb-OVA conjugates was determined by SDS-PAGE (10% gel) under non-reducing conditions. Functional activity of the CD40 mAb was checked by flow cytometric analysis on CD40 transfected fibroblast cells. Transfected or control cells were incubated with either the GL117 or 1C10 conjugate (10 μg/ml) for 20 min on ice. Following 3 washes with FACS buffer, samples were incubated with anti-OVA mouse serum at 1 in 100 dilution for 20 min on ice. Following a further 3 washes, samples were incubated with biotinylated anti-mouse-Ig for 20 min on ice then washed and incubated with streptavidin-PE. Negative controls included samples incubated with all secondary reagents in the absence of conjugates.

EDC Mediated Conjugation of mAb to Synthetic Peptides

An HIV gp120 derived synthetic antigenic peptide, shown to induce immunity (see The subunit and adjuvant approach, Hart et al M. F. Powell and M. J. Newman, Editors. (1995), Plenum Press: New York. p. 821-845. Conley et al Vaccine. 12: p. 445-451.) was selected for conjugation to anti-CD40 mAb for assessment of immunogenicity. This peptide, having the sequence CTRPNNNTRKSIRIQRGPG (SEQ ID NO: 1), was synthesised by Sigma-Genosys, UK.

Conjugations of peptide to mAb were carried out using EDC (1-Ethyl-3-(3-Dimethylaminopropyl) carbodiimide hydrochloride) obtained from Pierce (Rockford, Ill.). The reaction was carried out using a modified version the two-step protocol described in the manufacturer's instructions and is outlined below.

1C10 and control proteins (GL117 and ovalbumin) were dialysed overnight against activation buffer (0.1M MES, 0.5M NaCl, pH6.0) and peptides dissolved at 1 mg/ml in this same buffer. 0.4 mg of EDC (2 mM) was added to the peptide solution along with 1.1 mg (5 mM) NHS and reaction allowed to proceed for 15 min at RT. 1.4 μl of 2-ME was then added to quench the EDC. Anti-CD40 mAb or control proteins were then added to this reaction at a molar ratio of peptide to mAb of 1:1. Proteins were allowed to react at RT for 2 hrs. The reaction was stopped by addition of hydroxylamine at a final concentration of 10 mM. Samples were then extensively washed using 30 KDa cut-off centrifugal filters in PBS and the final protein concentration of conjugates determined by Bradford's method. Samples were then filter sterilised and stored at 4° C. until used.

Functional activity of CD40 mAb and presence of coupled peptide antigen was determined by flow cytometric analysis on CD40 transfected fibroblasts. Detection of bound peptide was achieved using a mouse anti-peptide antibody supplied by NIBSC.

EDC Mediated Conjugation of mAb to Recombinant HSVgD

1C10 and control mAb GL117 were dialysed overnight against conjugation buffer (50 mM phosphate, 1 mM EDTA) and then concentrated to 5 mg/ml using a 30 KDa cut-off centrifugal filter. Immediately prior to use, 6.5 mg of SATA (Sigma, UK) was dissolved in 500 μl DMSO. 1ml of the concentrated antibody solution was then incubated at RT for 30 min with 10 μl of the SATA solution. The reacted antibody solution was then washed three times over a 30 KDa cut-off centrifugal filter. Sulfhydryl groups introduced into the antibodies were then de-protected by incubating each mAb with 1001 μl of 0.5M hydroxylamine (in 50 mM phosphate, 25 mM EDTA, pH 7.5) per ml of antibody solution. This reaction was allowed to proceed for 2 hrs at RT. Meanwhile, maleimide activation of recombinant HSV gD (Viral Therapeutics). HSV gD was concentrated to 5 mg/ml in PBS and 1 mg of sulfo-SMCC added to 500 μl of the gD solution. Following 60 min incubation at RT, the maleimide activated gD was washed extensively with conjugation buffer, over a 30 KDa cut-off centrifugal filter. 400 μg of maleimide activated gD per mg of SATA reacted mAb were then mixed (made up to a final volume of 1 ml in conjugation buffer. This reaction was allowed to proceed for 1.5 hrs at RT and was stopped by the addition of 2-ME to a final concentration of 10 mM. The protein conjugate was then extensively dialysed against PBS, quantified by the Bradford assay, filter sterilised and stored at 4° C. until used.

Functional activity of CD40 mAb and presence of coupled herpes antigen was determined by flow cytometric analysis on CD40 transfected fibroblasts. Detection of bound glycoprotein D was confirmed using a mouse anti-HSV-1 antibody supplied by DAKO.

Conjugation of Synthetic CTL Peptide to Anti-CD40 mAb

The peptide (designated pHSV-CTL) is derived from HSV glycoprotein B (amino acids 498 to 505 having the sequence SSIEFARL (SEQ ID NO: 2). Peptide was synthesized by Dr. A. Moir (University of Sheffield, Department of Molecular Biology and Biotechnology).

Conjugation of peptide to mAb was carried out using the hetero-bifunctional cross-linker EDC (1-Ethyl-3-(3-Dimethylaminopropyl) carbodiimide hydrochloride) using a modified version of the two-step protocol described in the manufacturer's instructions (Pierce, Rockford, Ill.). 1C10 and control proteins (GL117 and ovalbumin) were dialysed overnight against activation buffer (0.1M MES, 0.5M NaCl, pH6.0) and peptides dissolved at 1 mg/ml in this same buffer. 0.4 mg of EDC (2 mM) was added to the peptide solution along with 1.1 mg (5 mM) NHS and reaction allowed to proceed for 15 min at RT. 1.41 μl of 2-ME was then added to quench the EDC. Anti-CD40 mAb or control proteins were then added to this reaction at a molar ratio of peptide to mAb of 1:1. Proteins were allowed to react at RT for 2 hrs. The reaction was stopped by addition of hydroxylamine at a final concentration of 10 mM. Samples were then extensively washed using 30 KDa cut-off centrifugal filters in PBS and the final protein concentration of conjugates determined by Bradford's method. Samples were then filter sterilised and stored at 4° C. until used.

A second conjugation experiment was performed with higher peptide to antibody ratios (5, 10, 20, 50 and 100 to 1), due to disappointing results obtained with the 1:1 conjugates. These reactions were carried out using the same protocol described above.

Analysis of mAb/Peptide Conjugates

The analysis of mAb/peptide conjugates prepared by EDC cross-linking was carried out by flow cytometric analysis on CD40 transfected and control fibroblast cells. The lack of anti-CTL peptide mAbs meant analysis could only be carried out using anti-rat mAbs (i.e. confirmation of anti-CD40 mAb binding). This was performed by incubating fibroblast cells with conjugate for 30 mins on ice, washing 3 times with FACS buffer and subsequent incubation with FITC labelled goat anti-rat antiserum (Pharmingen). Following a further 3 washes, samples were analysed using a FACSCalibur flow cytometer and CellQuest software.

SDS-PAGE analysis was also used to analyse conjugates, however this was found to provide no meaningful data on the size of conjugates.

SATA Conjugation of mAb to Heat Inactivated Influenza Virus

Antibodies were dialysed overnight against conjugation buffer (50 mM phosphate, 1 mM EDTA, pH 7.5), then concentrated to 5 mg/ml using a 30 KDa cut-off centrifugal filter. Immediately prior to use, 6.5 mg of SATA was dissolved in DMSO. 10 μl of this SATA solution was then added to each ml of the antibody solution, and incubated for 30 min at RT. The reacted antibody was then washed extensively, with conjugation buffer over a 30 KDa centrifugal filter. Meanwhile, the maleimide activation of heat inactivated influenza virus was proceeded with. HI virus stock (A/Bangkok/10/83) was quantified by Bradford assay and diluted to 8 mg/ml in conjugation buffer. 1 mg of sulfo-SMCC (sigma) was then added and the solution allowed to react for 1 hr at RT. The malieimide activated virus was then washed extensively over a 100 KDa centrifugal filter. The antibody and virus solutions were then combined, giving a range of virus:antibody ratios (10, 100 and 1000 mAbs per virion) and the reaction allowed to proceed for 1.5 hrs at RT. The reaction was stopped by addition of 2-ME (10 mM final conc.) and the conjugates dialysed, quantified and filter sterilised. Analysis of virus conjugates was carried out using flow cytometry on CD40 transfected fibroblasts. Detection of CD40-mediated influenza binding was determined using mouse anti-influenza serum.

Immunisation Protocols

Mice were treated with 500 μg of either 1C10, 4F11 or GL117 and 20 ng of PS3 i.p. except those receiving Pneumovax II. BALB/c mice receiving Pneumovax II were injected i.p. with either 500 μg of 1C10 or GL117 and 1/25^{th} of the recommended human dose of Pneumovax II. This equates to 1 μg of each of the 23 polysaccharides present in vaccine. At least 5 mice were used for each experimental group. In experiments where mice were immunised with avidin conjugated to biotinylated anti-CD40, avidin at 1 mg/1 ml and biotinylated antibody at 1 mg/ml were mixed together at a 1:1 ratio and left on ice for 30 minutes. The conjugates were then diluted in PBS to give a total of 10 μg antibody and 10 μg avidin in 0.2 ml PBS, which was then injected intraperitoneally. In cases where avidin in 0.3 ml PBS, which was then injected intraperitoneally. In cases where avidin alone was used it was pre-mixed with an equal volume of PBS and left on ice for 30 minutes before dilution and injection.

Four groups of five BALB/c mice were immunised with 10 μg of 1C10-OVA, GL117-OVA, 6 μg OVA alone or with 4 μg 1C10 and 6 μg OVA (calculated from the 1 to 1.5 reaction ratio) via intraperitoneal injection. 10 days after immunisation mice were bled via the dorsal tail vein and blood allowed to coagulate overnight at 4° C. Serum was then separated and stored at −20° C. until used. Serum levels of anti-OVA Ig from immunised mice were determined by ELISA on 96-well plates coated with OVA at 10 μg/ml in PBS.

Four groups of five BALB/c mice were immunised with 10 μg of 1C10-pHIV, GL117-pHIV, 10 μg of pHIV alone or with 10 μg of a 1C10/pHIV mix via intraperitoneal injection. 10 days after immunisation mice were bled via the dorsal tail vein and blood allowed to coagulate overnight at 4° C. Serum was then separated and stored at −20° C. until used.

Serum levels of anti-pHIV Ig from immunised mice were determined by ELISA on 96-well plates coated with peptide using a glutaraldehyde coupling technique. Perhaps the greatest consideration with this technique to ensure that only peptide specific antibodies are detected. Many coupling reactions lead to modifications of carrier protein residues and immunisation of animals with such conjugates results in production of CAMOR antibodies (coupling agent-modified residue). This is illustrated by Briand et al (1985) J Immunol. Methods 78: p59-69, where immunisation with a peptide coupled to BSA leads not only to specific antibodies for the peptide coupled to KLH, but the production of antibodies against irrelevant peptides coupled to KLH using the same coupling process. This phenomenon is also apparent with so called ‘zero length cross-linkers’ such as EDC. It is therefore important to use not only a different protein for the ELISA coating conjugate, but also a different coupling process. Peptide was coupled to fish gelatin as opposed to BSA as the latter is often a trace contaminant in purified mAbs and leads to anti-BSA responses in experimental animals. Coupling was carried out by coating 96-well plates with 5% fish gelatin overnight at 4° C. Plates were then washed and 50 μl of 0.4% glutaraldehyde and 50 μl of peptide (20 μg/ml) added, and plates incubated for 1 hr at RT. Plates were washed and 100 μl 0.5M ethanolamine added. Following 1 hr incubation at RT and subsequent washing, plates were blocked with 1% fish gelatin for 1 hr at RT. Standard ELISA techniques were then used for detection of peptide specific antigen.

Four groups of five BALB/c were immunised via the i.p. route with 10 g of mAb-HSVgD conjugate (anti-CD40 or control mAb), 10 mg of HSVgD/1C10mix (4 μg HSVgd/6 μg 1C10) or with 10 μg of HSVgD alone. Ten days after immunisation, mice were bled via the dorsal tail vein and serum separated following overnight incubation of the blood at 4° C. Serum anti-HSV titres were determined by standard ELISA techniques on EIA plates coated with HSVgD (10 μg/ml in PBS) overnight at 4° C.

Experiment in CD4 Depleted Mice

BALB/c mice, 6-10 weeks old, were depleted of CD4 cells 5 days before the experiment start. 500 μg of depleting anti CD4 antibody YTS 191.1 was injected intravenously and again the next day intraperitoneally. The percentage of CD4+ splenocytes in the depleted mice as detected by flow cytometry had dropped to undetectable levels when the antibody and PS3 were injected. There was no antibody response to 50 μg to keyhole limpet haemocyanin, a T dependent antigen, co-administered with the PS3 (data not shown).

Measurements of Polysaccharide Antibodies and Total Serum Immunoglobin by ELISA

96 well ELISA plates (Costar, UK) were coated overnight with 10 μg/ml polysaccharide or with a 1/200 dilution of anti mouse Ig serum (Sigma, UK). Individual sera were titrated on the plates and the various isotypes detected by HRP conjugated mouse isotype specific sera (Southern Biotechnology Associates, USA). Sera obtained from mice injected with Pneumovax II were absorbed against S. pneumoniae cell wall polysaccharide, a contaminant of all capsular polysaccharide preparations might have created false positive results. Total serum immunoglobulin concentrations were calculated with reference to calibrated mouse serum (Sigma, UK). With the polysaccharide results end point titres for each mouse were assessed against normal mouse serum and then geometric mean titres and standard deviation calculated.

Measurement of Anti-avidin Responses by ELISA

96 well ELISA plates (Costar, UK) were coated overnight with 10 μg/ml avidin (Sigma) in PBS. After blocking for 1 hour with 3% bovine serum albumin individual sera were titrated on the plates, incubated at room temperature for 1 hour, and following washing, antibody was detected using HRP conjugated anti-mouse immuglobulin (Southern Biotechnelogy Associates USA), and substrate (OPD Sigma). End point titres for each mouse were assessed against normal mouse serum, and then geometric mean titres and standard deviation calculated.

Challenge with S. pneumoniae

BALB/c mice were immunised 9 months before challenge with 20 ng PS3 and 500 μg 1C10 i.p. Challenge was 10⁵ colony forming units of encapsulated S. pneumoniae type 3 (ATCC) given i.p. Final numbers surviving were ascertained 2 weeks after challenge.

Assessment of Anti-CD40 mAb Toxicity

Groups of 6 female BALB/c mice were injected via the intraperitoneal route with 200 μl (in PBS) of the anti-CD40 mAb 1C10, or isotype matched control antibody, GL117, at a range of concentrations (500 μg to 1 μg per mouse). Five days after immunisation, three mice from each group were sacrificed by cervical dislocation and spleens removed and weighed. Mean spleen weights for each group were then calculated. Ten days after the initial immunisation, the remaining three mice were bled via the dorsal tail vein and serum collected from blood samples after overnight coagulation at 4° C. Serum was stored at −20° C. until used for polyclonal Ig quantification.

Polyclonal Ig responses in mAb immunised animals were determined using an ELISA based assay. Plates were coated overnight at 4° C. with goat anti-mouse Ig at 10 μg/ml (Jackson ImmunoResearch Laboratories). A mouse Ig standard (Sigma) was then applied to the plate (5 μg/ml) and doubling dilutions of this sample made across the plate. Test serum samples were then applied to the plate, starting at a 1 in 10 dilution, and tenfold dilutions made across the plate. Total serum Ig in samples was calculated via extrapolation from the mouse Ig standard curve. To ensure that this system did not detect any possible residual rat antibody from the immunisation, the anti-CD40 mAb 1C10 was included as a control sample. No detection of 1C10 was apparent in the system.

### EXAMPLE 1

The development of vaccines against encapsulated bacteria, such as Streptococcus pneumoniae, Haemophilus influenzae and Neisseria meningitidis, is centred on their distinctive capsular polysaccharides. Unfortunately, the inability of antigen presenting cells (APC) to process and present polysaccharides with MHC class II means that these antigens cannot stimulate T-cells. Polysaccharide specific B-cells receive no direct help from their T-cells and, therefore, these antigens are considered T independent (TI-II). Due to this lack of help TI-II antibody responses are of low titre, low average affinity, and are predominantly of the IgM class with no boosting on second or later exposures to antigen. The T-cell help provided during immune responses to TD antigens induces high titre and isotype switched antibody responses. The major stimulus to B-cells is provided by CD154 (formerly CD40 ligand or gp39), which is expressed de novo on activated T-cells. The CD154 molecule binds the CD40 antigen, which is constitutively expressed on B-cells, and their interactions provide key signals as immune responses develop. CD40 activation is important for the initiation of B-cell proliferation, immunoglobulin class switching, germinal centre responses, and the production of memory B-cells and plasma cells. B-cells responding to TI-II antigens lack T-cell derived cytokines and CD40 litigation and produce, as a result, the poor antibody response characteristic of TI-II antigens. We have investigated in vivo whether the administration of pneumococcal polysaccharide with anti-mouse CD40 antibody could provide a substitute for CD154 mediated CD40 litigation. The two antibodies used were 1C10 and 4F11, chosen they are both rat IgG2a anti-mouse CD40 antibodies but possess markedly different in vitro properties.

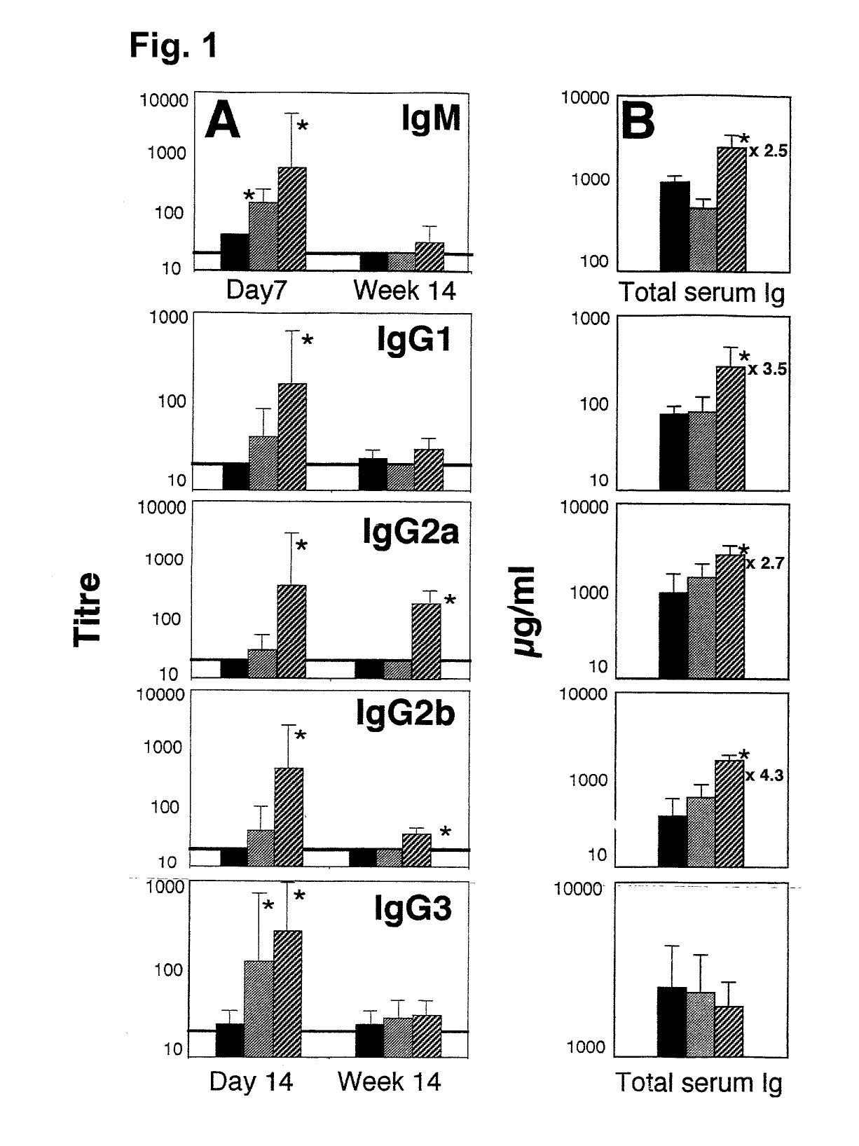

Intraperitoneal immunisation of BLAB/c mice with type 3 pneumococcal capsular polysaccharide (PS3) alone induced weal IgM and IgG3 responses against the antigen (FIG. 1A). This is typical of the response to TI type II antigens in mice (humans produce IgM and IgG2). Administration of antibodies 1C10 or 4F11 with PS3 induced small but significant rises in specific IgM and IgG3, while remarkably, 1C10 induced significant polysaccharide specific IgG1, IgG2a and IgG2b responses. These isotopes are not normally seen in response to TI II antigens. 1C10 would appear to have successfully mimicked T-cell help by inducing high antibody titres and isotype switching in vivo. The anti-polysaccharide response was extremely persistent, with antibody being detected at high titres 14 weeks after the single immunisation (FIG. 1A). No memory response against the polysaccharide was induced as a second injection of polysaccharide along failed to boost antibody responses (data not shown).

### EXAMPLE 2

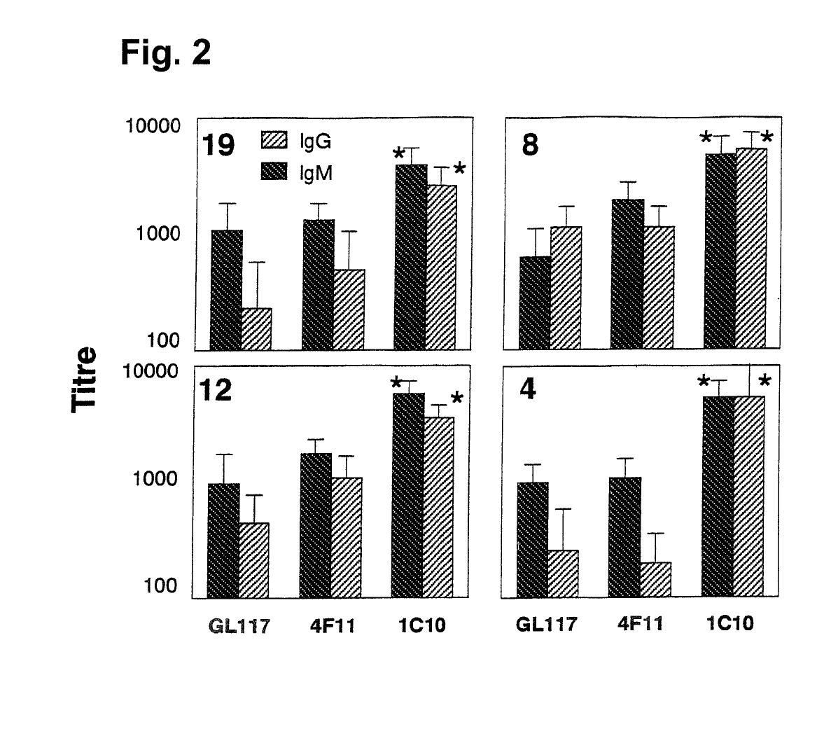

S. pneumoniae has over 80 different capsular polysaccharide types and any vaccination would be expected to induce protective immunity against a number of more common stereotypes. A current pneumococcal vaccine, Pneumovax II (Merck, Sharp and Dohme), consists of 23 different polysaccharides. Mice were immunised with this 23-valent vaccine and 1C10. FIG. 2 shows that inclusion of the CD40 antibody successfully generated strong IgG responses against randomly chosen polysaccharide types 4, 8, 12 and 19. Such isotype switched responses were also generated against the two other antigens were examined, types 3 and 14 (data not shown). Therefore, 1C10 enhances responses to TI-II antigens other than just PS3.

### EXAMPLE 3

Given that administration of CD40 antibody mixed with polysaccharide would not restrict or even target CD40 ligation to antigen specific B-cells, we anticipated polyclonal activation of B-cells with a resultant rise in total serum immunoglobulin levels. Indeed 1C10 and PS3 induced some splenomegaly and 2-4 fold rises in total serum immunoglobulin levels (FIG. 1B). This, however, should be contrasted with up to 5-fold rises in specific antibody levels, indicating that polysaccharide specific antibody production was preferentially enhanced. This skewing towards specific antibody is also not unexpected as it reflects in vitro findings. In vitro, while 1C10 could induce B-cell proliferation in the absence of stimulation through the antigen receptor, proliferation was synergistically enhanced by such co-stimulation. 4F11, which largely lacks agonist activity in vitro, did not enhance responses as efficiently as 1C10, demonstrating an association between adjuvant activity in vivo and B-cell activation in vitro.

### EXAMPLE 4

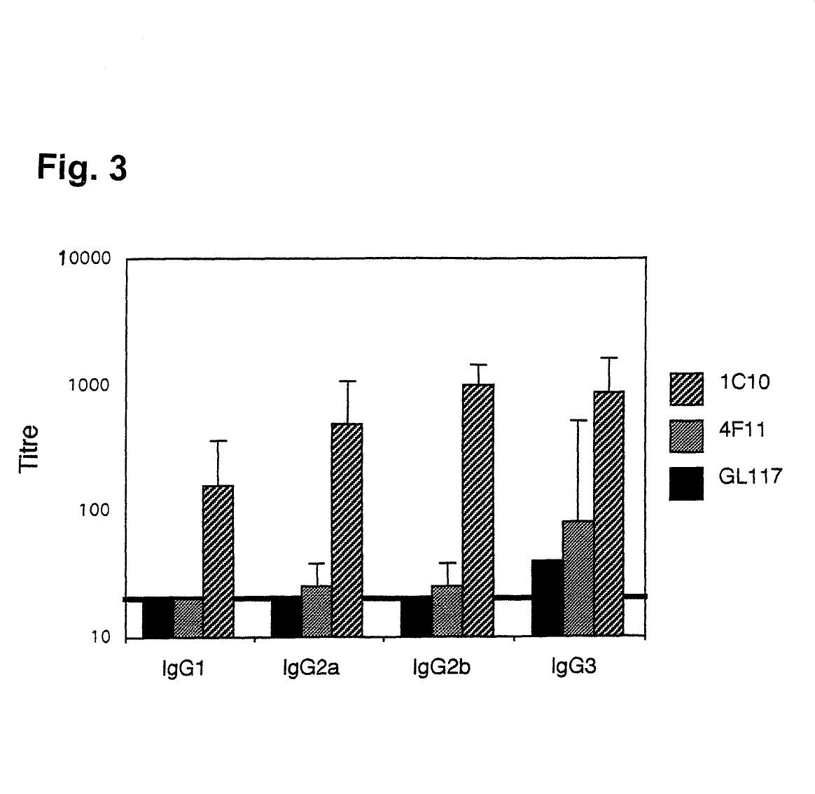

CD40 ligation is necessary for switching to IgG isotypes during a T dependent response, but various cytokines also play important roles. It was, therefore, intriguing that such isotype switched responses were obtained without the addition of exogenous cytokines. This suggests either that CD40 and antigen receptor ligation may be sufficient to induce isotype switching or that bystander cells may provide sufficient cytokines to switch the activated B-cells in vivo. We considered that the CD40 antibodies might be stimulating T-cell production, whether directly through ligation of CD40 on T-cells or indirectly through induction of co-stimulatory molecules on B-cells or other APCs. The action of 4F11 showed T-cell dependency as it failed to augment polysaccharide specific responses in CD4 depleted mice (FIG. 3) with IgG responses to polysaccharide being better than those induced in normal mice, demonstrating a CD4 independent action. Similar results were obtained when athymic nude mice were used instead of CD4 depleted mice (data not shown).

### EXAMPLE 5

Most vaccines under development for use against encapsulated bacteria are protein-polysaccharide conjugates which aim to provide T-cell help for the anti-polysaccharide response through T-cell recognition of epitopes on the protein. By their nature such conjugates are not as effective in CD4 deficient patients such as those with AIDS. In contrast the use of a CD40 stimulator would not only avoid the high cost of conjugate production, but as we have shown, generate responses unaffected by a CD4 deficiency.

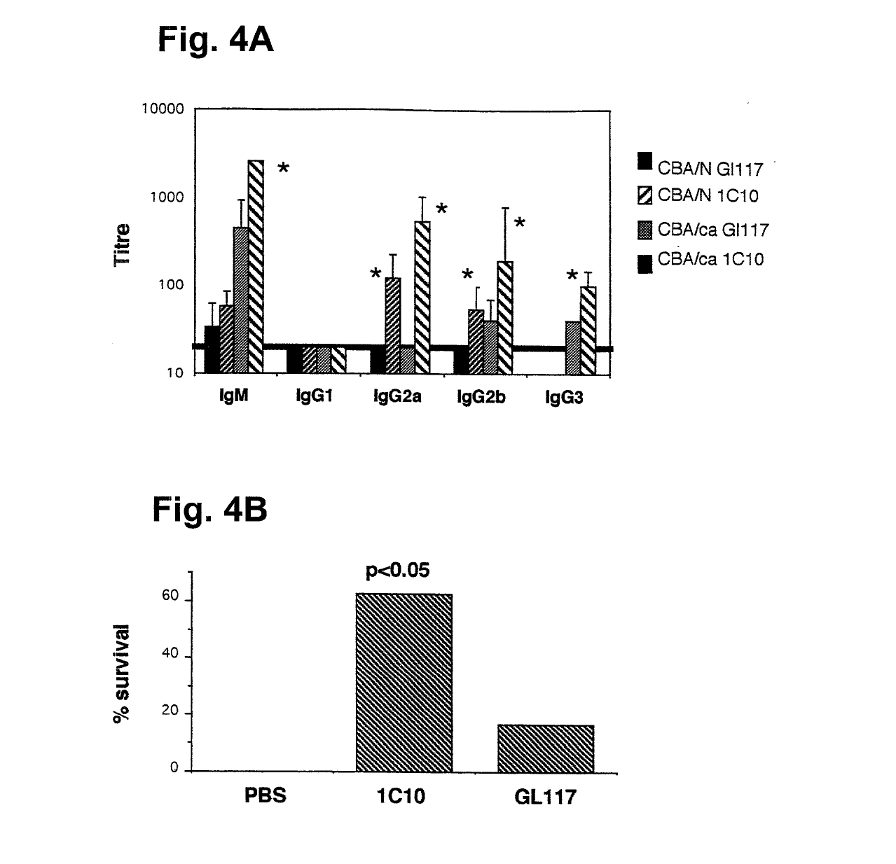

The major fault with capsular polysaccharide only vaccines is that infants and young children, whilst reacting normally to TD antigens, respond poorly to TI-II antigens. Indeed children under two years old fail to respond at all to many TI-II antigens. The inability of their immune systems to act against bacterial capsules correlates with increased susceptibility to infection. They are the group most in need of effective vaccines. CBA/N (xid) mice have an X-linked immunodeficiency rendering them, like infants, unable to respond to TI-II. Although one report has stated otherwise, in our hands these mice react normally to CD40 litigation in vitro (and unpublished data A.H>). We immunised groups of xid mice with 1C10 plus PS3 and successfully generated IgG2a and IgG2b responses against PS3 (FIG. 4A). Thus, the B-cell defect in these mice was successfully by-passed by administering the CD40 antibody as an adjuvant along with antigen.

Using the mouse model system, we have shown that CD40 simulators can enhance the antibody response to pneumococcal polysaccharides, producing greater antibody levels and the production of IgG isotypes. Similar to protein-polysaccharide conjugates, 1C10 can induce polysaccharide specific responses in xid mice, with like infants are unable to respond to polysaccharide only based vaccines. Unlike protein-polysaccharide conjugates, the adjuvant action of 1C10 is CD4 cell independent, which is a definite advantage for the vaccination of patients with CD4 deficiencies, for example AIDS sufferers.

While 1C10 administered with PS3 clearly enhances specific antibody responses, the measure of a vaccine is whether it provides long-term protection against disease. We challenged mice, immunised 9 months previously, with 10⁵ CFU of S. pneumoniae type II (FIG. 4B). Of the BALB/c mice administered with PS3 and 1C10 five of eight survived challenge, whereas only one of sic and none of eleven mice survived in the groups receiving, respectively PS3 with GL117 and PS3 alone (p<0.05 χ² test).

### EXAMPLE 6

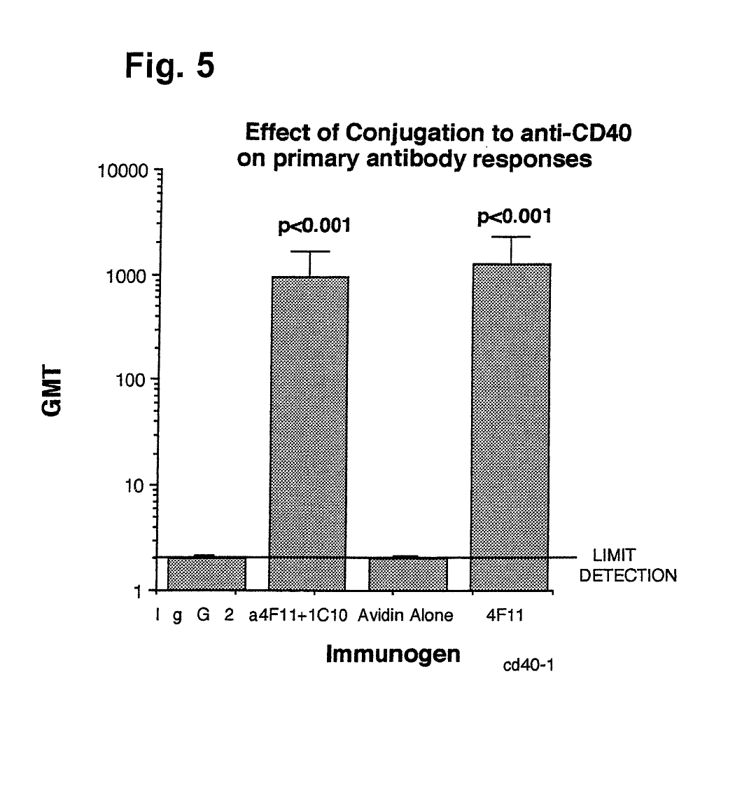

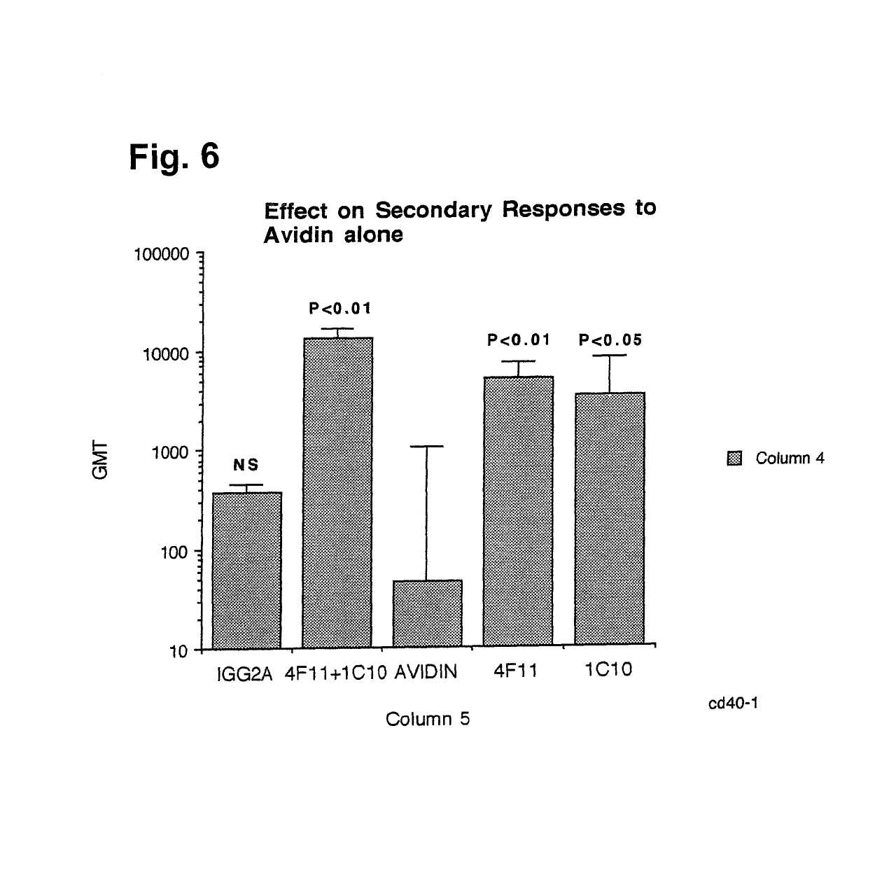

The induction of polyclonal antibody responses, as previously described in FIG. 1B, may increase the risk of auto antibody production. We have investigated this problem by reducing the need to administer elevated doses of anti CD40 antibody by conjugating biotinylated anti CD40 antibody with avidin (a natural ligand of biotin). By physically linking the adjuvant and antigen we have been able to reduce adjuvant levels by approximately 50-fold. FIG. 5 shows the primary responses of BALB/c mice to a combination of biotinylated 4F11 and 1C10 conjugated with avidin, to biotinylated 4F11 conjugated to avidin or to avidin alone. The primary antibody response to avidin is comparable to the response to avidin plus biotinylated IgG2a control antibody. However significant enhancement of antibody levels to avidin is achieved in response to immunisation with biotinylated anti CD40/avidin conjugate. FIG. 6 shows secondary antibody responses. Clearly the physical linkage of antigen to adjuvant leads to enhanced antibody responses to avidin with a reduction in the amount of adjuvant required. This methodology may also be applied to T-cell independent antigens like the capsular polysaccharides of S. pneumoniae. Techniques for conjugating polysaccharides to protein do exist and will allow this strategy to be further developed.

It is evident that CD40 simulators, such as antibodies, recombinant soluble CD154, or molecular mimics of CD154, have considerable potential as immunological adjuvants for T-cell dependent/independent antigens.

### EXAMPLE 7

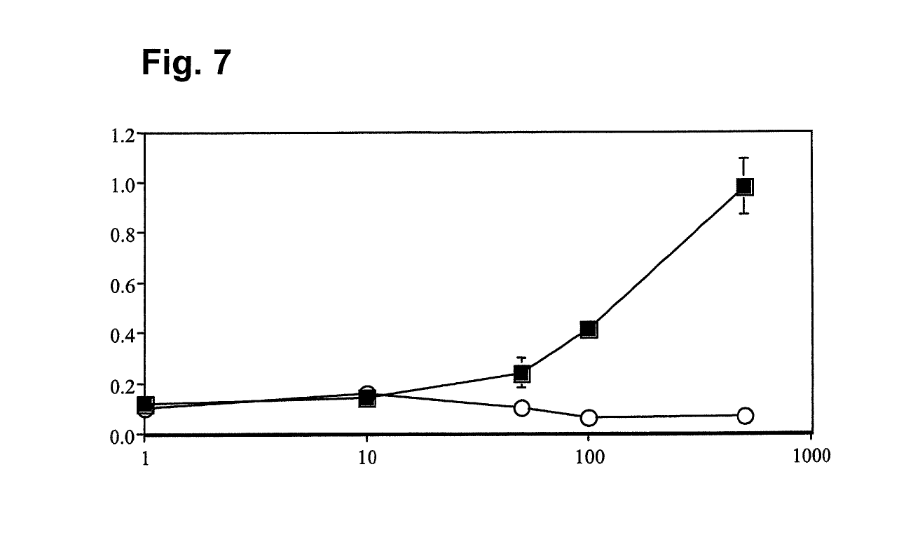

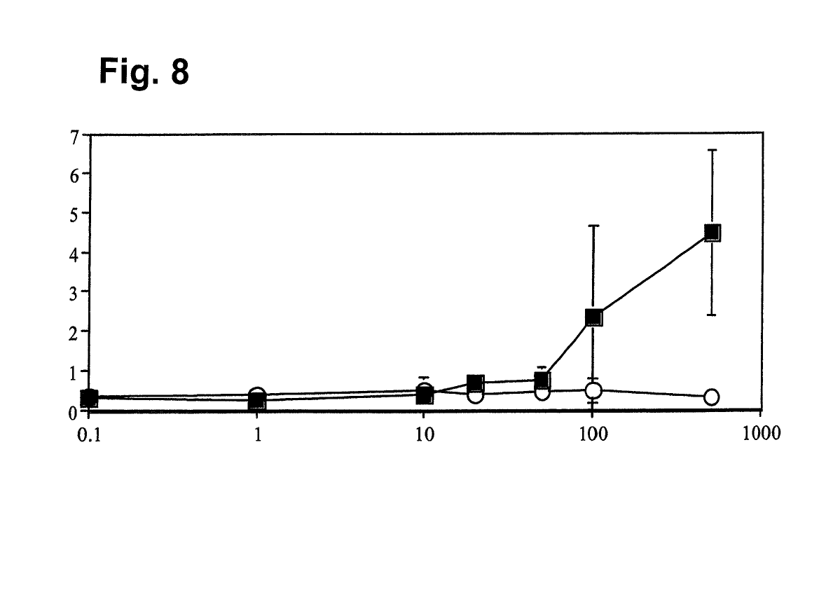

A major problem with many experimental adjuvants is toxicity which may be caused by induction of cytokine release or other mechanisms leading to activation of non-antigen specific lymphocytes and other immune cells. Such undesirable side-effects can be detected in a number of ways. Polyclonal activation of non-antigen specific lymphocytes can be detected by increased cell numbers, leading to swelling of secondary lymphoid organs, such as the spleen. Polyclonal stimulation of non-antigen specific B cells may give rise to an increase in total serum immunoglobulin levels. FIG. 7 shows spleen weights of mice 5 days after injection with anti-CD40 or an isotype control antibody at various doses. Spleen weights were significantly increased at doses of antibody from 500 ug down to 50 ug. FIG. 8 shows that total serum immunoglobulin 10 days after anti-CD40 administration was increased at doses down to 100 ug.

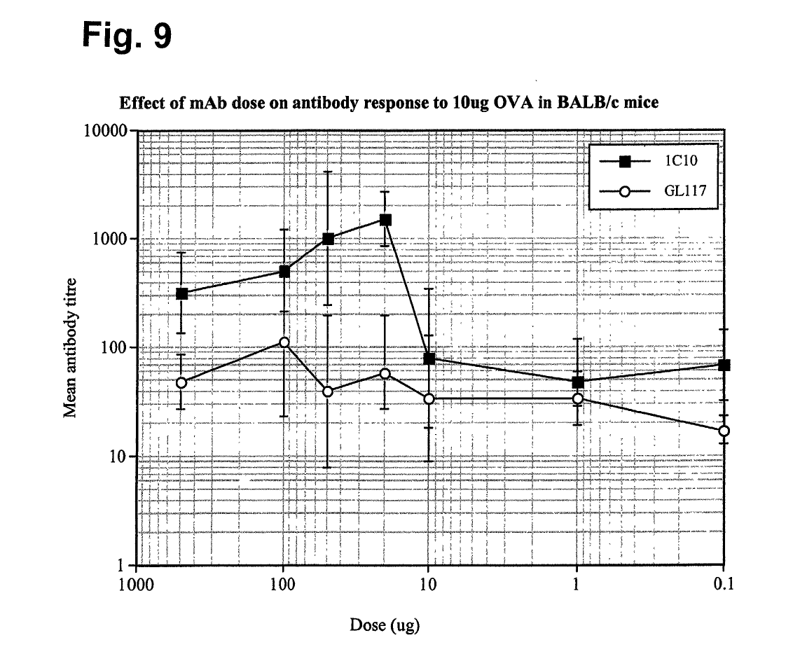

The adjuvant effect of anti-CD40 mixed with antigen correlated with these toxic effects. FIG. 9 shows the antibody response to ovalbumin induced by co-administration of ovalbumin with anti-CD40 or control antibody at doses from 500 ug to 0.1 ug. The adjuvant effect of anti-CD40 is not evident at doses below 50 ug.

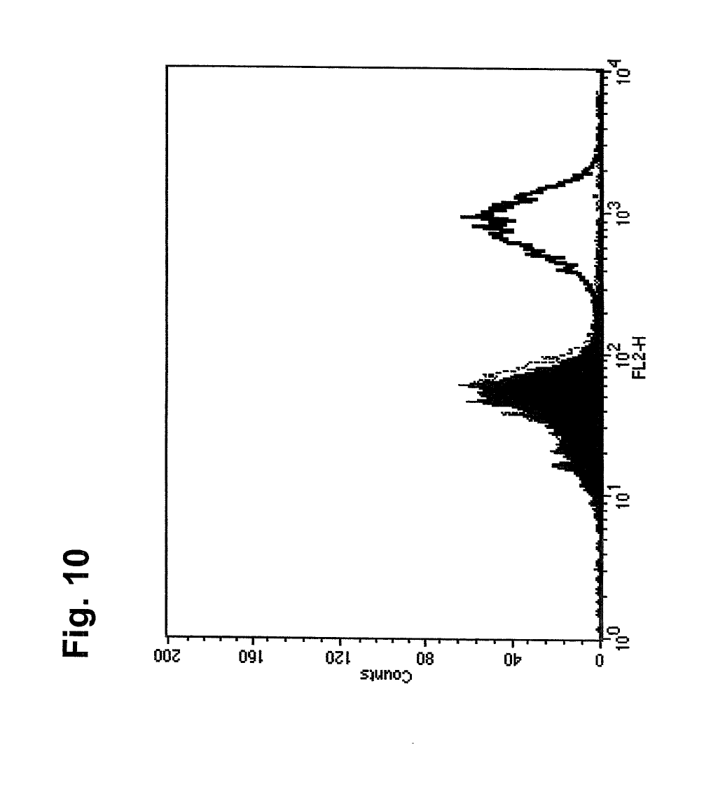

Coupling of anti-CD40 to antigen disconnects the adjuvant effect from the toxicity. Thus, as shown in FIG. 10 the adjuvant effect of CD40 antibody attached to antigen, as assessed by measuring anti-rat IgG2a responses (the CD40 antibody is a rat antibody, and thus acts as an antigen coupled to the CD40 binding region in this case) is strongly enhanced at anti-CD40 doses down to only lug per mouse. Toxicity is not evident, while the adjuvant effect remains very strong, in fact it is stronger than that of the mixture. The isotype control antibody in this case is also rat IgG2a, and so this acts as the same antigen, lacking CD40 binding.

An important point is that the enormous enhancements in antibody responses are seen after only a single immunisation with CD40 conjugates. Achieving high levels of immunity with one immunisation is a major aim of the W.H.O as there are enormous cost and social benefits to be had from cutting the number of visits to the clinic required.

| Immunisation | Avidin | Ovalbumin | Rat IgG2a |
|---|---|---|---|
| Avidin | 1.23 (0.39) | 1 (0) | 1 (0) |
| Avidin + | 4.66 (0.13) p > 10⁻⁶ | ND | 4.51 (0.44) p > 10⁻⁶ |
| biotinylated 1C10 | | | |
| Avidin + | 2.2 (0.59) NS | ND | 2.1 (0.26) NS |
| biotinylated GL117 | | | |
| Avidin + strepavidin + | 1.45 (0.89) NS | ND | 4.53 (0.24) p > 10⁻⁶ |
| biotinylated 1C10 | | | |
| Avid + unbiotinylated | 1.59 (1.34) NS | ND | 4.53 (0.24) p > 10⁻⁶ |
| 1C10 | | | |
| Ovalbumin | ND | 1 (0) | 1.05 (0.13) |
| Ovalbumin + | ND | 1 (0) | 4.49 (0.16) p > 10⁻⁶ |
| biotinlayted 1C10 | | | |
| IgG2a control 20 μg | ND | ND | 3.53 (0.49) |
| 1C10 20 μg | ND | ND | 6.41 (0) p > 10⁻⁶ |
| Ovalbumin + | ND | 1 (0) | |
| 0.5 mg GL117 | | | |
| Ovalbumin + | ND | 2.8 (0.93) p > 0.01 | |
| 0.5 mg 1C10 | | | |

ND = Not determined
NS = Not significant

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations falling within the scope of the appended claims and equivalents thereof.

What is claimed is:

1. A method of reducing the toxicity of an adjuvant to an animal comprising the steps of:
i) providing an adjuvant preparation comprising a CD40 ligand and at least one antigen wherein said CD40 ligand is crosslinked to said antigen;
ii) administering an effective amount of the crosslinked adjuvant to said animal sufficient to provoke an immune response to said antigen by activation of a CD40 presenting cell.

2. A method according to claim 1, wherein said CD40 ligand is an antibody, or binding part thereof, which binds CD40.

3. A method according to claim 2, wherein said antibody is a monoclonal antibody.

4. A method according to claim 3, wherein said monoclonal antibody is humanised.

5. A method according to claim 1, wherein said CD40 ligand is CD40L.

6. A method according to claim 1, wherein said adjuvant activates a CD40 presenting B-lymphocyte to promote immunoglobulin secretion.

7. A method according to claim 1, wherein said adjuvant activates a CD40 presenting B-lymphocyte to promote immunoglobulin isotype switching.

8. A method according to claim 1, wherein said antigen is a T-cell dependent antigen.

9. A method according to claim 8, wherein said T-cell dependent antigen is a viral antigen.

10. A method according to claim 9, wherein said viral antigen is an HIV antigen.

11. A method according to claim 10, wherein said HIV antigen is a polypeptide comprising the amino acid sequence CTRPNNNTRKSIRIQRGPG (SEQ ID NO: 1).

12. A method according to claim 8, wherein said viral antigen is a herpes simplex virus antigen.

13. A method according to claim 12, wherein said herpes simplex virus antigen is a glycoprotein.

14. A method according to claim 13, wherein said glycoprotein is glycoprotein D.

15. A method according to claim 13, wherein said glycoprotein is glycoprotein B.

16. A method according to claim 15, wherein said glycoprotein comprises the amino acid sequence SSIEFARL (SEQ ID NO: 2).

17. A method according to claim 9, wherein said antigen is an influenza antigen.

18. A method according to claim 17, wherein said antigen is derived from influenza isolate A/Bangkok/10/83.

19. A method according to claim 18, wherein said antigen consists of influenza isolate A/Bangkok/10/83.

20. A method according to claim 1, wherein said antigen is a T cell independent antigen.

21. A method according to claim 20, wherein said T-cell independent antigen is a polysaccharide.

22. A method according to claim 21, wherein said polysaccharide are capsular polysaccharides of bacterial species selected from the group consisting of: Streptoccocus pneumoniae, Haemophilus influenzae and Neisseria meningitidis.

23. A method according to claim 22, wherein said capsular polysaccharides are derived from Streptoccocus pneumoniae and selected from the group consisting of: type 1, 3, 4, 8, 12, 13, 19 or 23.

24. An adjuvant comprising a CD40 ligand crosslinked to at least one viral antigen.

25. An adjuvant according to claim 24 wherein said viral antigen is an HIV antigen.

26. An adjuvant according to claim 25 wherein said viral antigen is a polypeptide comprising the amino acid sequence CTRPNNNTRKSIRIQRGPG (SEQ ID NO: 1).

27. An adjuvant according to claim 24 wherein said viral antigen is a herpes simplex virus antigen.

28. An adjuvant according to claim 27 wherein said herpes simplex virus antigen is glycoprotein D, accession number NP044668.

29. An adjuvant according to claim 27 wherein said herpes simplex virus antigen is glycoprotein B.

30. An adjuvant according to claim 29 wherein said glycoprotein B comprises the amino acid sequence SSIEFARL (SEQ ID NO: 2).

31. A vaccine composition comprising an adjuvant according to claim 24.

32. A vaccine composition comprising an adjuvant according to claim 25.

33. A vaccine composition comprising an adjuvant according to claim 26.

34. A vaccine composition comprising an adjuvant according to claim 27.
